(19) United States
(12) Patent Application Publication
(10) Pub. No.: US 2015/0165165 A1
(43) Pub. Date: Jun. 18, 2015

(54) EXTRACTION DEVICE FOR COLLECTING BLOOD SAMPLES, INCLUDING A CATHETER AND A SAFETY SYSTEM

(71) Applicant: EQUIPOS MEDICOS VIZCARRA, S.A., Emiliano Zapata, OT (MX)

(72) Inventors: Jose Maria Rodriguez Lelis, Emliano Zapata (MX); Jose Antonio Arellano Cabrera, Emiliano Zapata (MX); Maria Teresa Lucas Jimenez, Emiliano Zapata (MX); Alfredo Chalita Vizcarra, Emiliano Zapata (MX)

(21) Appl. No.: 14/412,352

(22) PCT Filed: Jul. 4, 2013
(86) PCT No.: PCT/MX2013/000079

§ 371 Date: Dec. 31, 2014

(30) Foreign Application Priority Data
Jul. 4, 2012 (MX) MX/A/2012/007851

(51) Int. Cl. A61M 25/06; A61B 5/15; A61B 5/153

(52) U.S. Cl. CPC A61M 25/0606; A61B 5/153; A61B 5/150992; A61B 5/150389; A61M 25/065; A61B 5/150351; A61B 5/150595

(57) ABSTRACT

The Extraction Device for Collecting Blood Samples, including a Catheter and a Safety System in which a flexible catheter instead of a metallic cannula with sharp edges for extracting blood is presented. This catheter is inserted through a traditional cannula, which retracts once the catheter is inside the vein, by means of a spring and safety latch mechanism, retaining the cannula within a rigid housing. The retracted cannula remains immobile within the rigid housing thus avoiding reutilization of the devise and accidental puncture wounds during its disposal. This EDCBSCSS has the advantages of (a) reducing it) or eliminating the use of a metallic cannula with sharp edges, which remains inside the vein during the entire procedure; (b) using a plastic cannula or catheter for extracting the blood sample which provides greater comfort for the patient and the assurance that the cutting element has been disabled; (c) possessing a safety system for medical personal so that after the procedure no accidental puncture wounds occur and (d) being easy to handle. The EDCBSCSS is made up of three sequential operational systems, which are the puncturing and retracting system which are located within housings (4) and (5) and are comprised of (1) a puncturing cannula; (2) a catheter; (3) a catheter space, (9) a spring and (8) a safety latch.

### FIELD OF THE INVENTION

The present invention is related to the hospital instruments manufacturing industry. More specifically it is related to the industry of manufacturing instruments for extracting and collecting blood. Still more specifically, it is related to the industry of manufacturing instruments for extracting blood safely, by means of a needle-catheter system which prevents the accidental puncturing or cutting of the vein by replacing the needle for a plastic catheter without sharp edges, which also prevents accidental punctures following its use.

### BACKGROUND OF THE INVENTION

Phlebotomy is the process of making an incision in a vein and is generally related to venipuncture and the extraction of blood. For decades phlebotomy has been and today it is one of the most common invasive techniques in health care [1]. There are two types of systems used for collecting blood: (a) open systems in which a blood sample, once it has been extracted for the patient's vein, must be transferred to another container for later analysis, these systems including hypodermic needles and syringes; (2) closed systems in which the blood that is collected is stored in the final container for later analysis and in which there is no need for transferring the blood to another device. Closed systems for the extraction of blood are preferred over the open systems due to their safety [2].

In 1943 [3] the American Red Cross asked a business which manufactured hospital material to develop a disposable, sterile apparatus for extracting blood. Once bottled, the material had to be kept sterile for use in battlefields. The result was the creation of a vacuum device, which allowed for extraction of blood directly from the vein, using a double pointed needle. One of the tips was directly connected to the collecting tube, which was in a vacuum, while the other tip of the needle was inserted into the vein. The blood was sucked into the vacuum tube, constituting a vacuum system for extracting blood. Since its beginnings, this device has been improved and perfected, transforming the system for blood extraction into a safe, practical procedure, which provides better quality for the diagnostic model. Today this kind of device for extracting and collecting blood samples is very common.

On the other hand, there is worldwide epidemiological evidence that shows that the main concern about accidents in the use of instruments with sharp points and their disposal as infectious waste in hospitals is the transmission of the AIDS virus and more commonly Hepatitis B and C viruses. This is due to the wounds caused by needles contaminated with human blood. Therefore there has been a growing need for producers of devices for extracting blood to try to find systems, which prevent accidental puncture wounds with needles to the medical staff and other workers who handle hospital waste.

As a result, new products are designed to incorporate special covers for needles or mechanisms for retracting the needle into a protective chamber. Such devices are described, for example, in U.S. Pat. Nos. 5,356,392, 6,004.278, 6,102.894, 6,186.960, US 2003/208.161 A1, US 2005/1187,493 A1, Pat. No. 7,513,887 B2, US 2010/241,029 A1, US 2011/166,476 A1. All of the devices described in these patents involve the use of a hollow needle, which punctures the patient's vein in order to obtain a blood sample and remains in the vein for the duration of the procedure. This process does not prevent the patient from being wounded by the movement of the needle within the vein and the only safety provided is after extracting the needle from the patient for final disposal as hazardous wastes.

The continuous use of metal cannulas, which remain in the vein during blood extraction, causes irritation to the inside of the vein by the pointed end of the cannula. This can be dangerous since phlebitis may occur and in some cases, excessive damage to the vein nullifies the procedure. Today, blood extraction is performed manually, making it necessary for the person who performs the extraction to use very refined techniques and in most cases special training in the use of the devices used as well as broad experience in order to avoid harming and thus affecting the patient when blood extraction is carried out. However, even with experience and expertise, the patient is often affected by pain, tissue damage, multi-punctures and general discomfort.

One method developed to avoid the need for keeping a metallic cannula with sharp tips in the vain is the use of plastic catheters, which have disposable needles which rim through the catheter to puncture the vein and allow the insertion of a catheter. Later, the needle may be removed, leaving the catheter in place to use as a connection for an intravenous bag, bottle or stopper for later use. With this method there is a flexible object with no sharp points inside the vein, thus preventing excessive irritation to the walls of the vein and eliminating the possibility of damaging the vein with a sharp object and the consequential ineffectiveness of the procedure. Moreover, psychologically the stress of the patient and the medical staff is diminished with the use of a non-cutting point, producing an improved procedure and greater comfort for the patient.

In patents such as US patent 2003/208,161 A1 can be found a design for devices that ensure that the cannula loses its sharp edges after use. This consists of two concentric nested tubes, in which one has a sharp tip and the other a tip that is not sharp. Here the sharp tip is counterposed to the point which is not sharp, so that during the puncturing procedure the sharp tip cuts the skin and when it enters the vein the inner tube is deployed so that it blocks the sharp tip of the exterior tube exposing a tip which is not sharp. This mechanism affords the vein with a degree of safety of not being damaged by the tip of the needle, but there is still a rigid metallic element present which may tear the vein.

As may be inferred from the text, there are certain characteristics and qualities that a device for extracting and collecting blood must possess, among them: (a) reduce or eliminate the use of a metallic cannula with sharp edges and which remains in the vein for the duration of the procedure; (b) the use of a plastic cannula or catheter for the extraction of blood samples which provides the patient greater comfort with the assurance that the sharp element has been disabled; (c) possess a safety system for medical personnel that assures that following the procedure, no accidental puncture wounds occur and (d) be easily handled. These are the characteristics claimed in the present invention.

### OBJECTIVES OF THE INVENTION

The main objective of the invention is to achieve a device for the extraction of blood, which avoids prolonged permanence of a cannula with sharp edges inside the vein, replacing the latter with a flexible plastic catheter, which avoids damaging the patient's vein.

Another objective is to have a safety system, which avoids accidental puncture wounds to medical personnel and janitorial workers, with a hollow needle contaminated with blood by way of confining the used needle within a rigid casing.

Still another objective is to have a device for collecting blood samples with ergonomic geometry and which is easy for the personnel in charge of carrying out the procedure of extracting and collecting blood to use.

And all of those objective and advantages which will become apparent upon reading the attached drawings which for illustrative, with non-limiting purposes, make up an integral part of the present description.

### BRIEF DESCRIPTION OF THE INVENTION

The present invention refers to a device for extracting and collecting blood samples, specifically to a system for extracting blood samples which replaces the use of a hollow needle with a flexible plastic catheter for extracting blood from the vein of a patient and in which the hollow needle after making the hole and inserting the catheter is confined in a rigid casing so that the device cannot be reused and also thus preventing accidental puncture wounds to medical personnel or others with the hollow needle contaminate with blood, thereafter proceeding to safe elimination of the device.

This invention refers to a double system which allows, through one of its components, a system for extracting blood samples which uses a flexible catheter without a point which remains in a patient's vein, thus avoiding wounds to the vein and in which the hollow needle is only used for inserting the catheter into the patient's vein and will be immediately withdrawn, removing the sharp tip from the vein. In the second component, a safety mechanism which once the procedure of extracting blood is finished, allows for withdrawing the contaminated cannula into a protection chamber thus avoiding later contact with the user or other persons, preventing accidental puncture wounds and the transmission of diseases.

Moreover, with a system which utilizes a flexible catheter instead of a hollow needle for taking blood samples, the stress produced by a sharp metallic object within the body is reduced, thus preventing serious wounds which may invalidate the procedure. The procedure with the use of a flexible catheter gives medical personnel greater confidence when carrying out blood extraction and gives patients more comfort.

Furthermore, the characteristics of the retracting system of the device assure there is no contact with the cannula after placing the catheter, and allows for its being placed at a safe distance within the protection chamber of the safety system.

The mechanism consists of two spaces joined at the ends of a hollow housing and in which one of the spaces holds a flexible catheter and the other space, located at the opposite end of the space of the catheter, holds a rubber cap. A cannula or hollow puncturing needle passes through the flexible catheter, which is connected to a safety latch located within the main housing. In the latch and on the opposite side, the puncturing cannula is connected to another cannula or hollow collecting needle which permits the flow of blood towards the container tubes. The safety latch is fixed, by means of a safety lock head to the housing in a primary slot and on slide guides of the housing and under pressure by a compressed spring in the front part, in the position called the charge state. Once the puncturing is carried out and the catheter and cannula are inserted into the vein, the safety latch is disconnected manually from the primary slot and, due to the decompression of the spring, it slides inside the housing until the head of the safety latch is fixed in the secondary slot. This process causes the puncturing cannula to withdraw from the vein and remain within the main housing, and the collecting cannula is now located within the rubber cap. The configuration catheter-puncturing cannula-safety latch-extraction cannula creates a flow channel to lead blood to be deposited once the vacuum collecting tube is in place.

Once the process of collecting blood samples is completed, the catheter of the device is removed from the vein leaving only a flexible component, without a point, exposed, while the cannula or hollow needle is confined within the rigid housing, thus avoiding any accidental puncture wounds which could occur to anyone who has contact with the device during the disposal process.

### BRIEF DESCRIPTION OF THE DRAWINGS

FIG. 1 shows an isometric view of the Extraction Device for Collecting Blood Samples including a Catheter and Safety System, from here on out called EDCBSCSS.

FIG. 2 is an exploded drawing showing the parts that make up the EDCBSCSS.

FIG. 3 shows the details of the protective tube and primary housing of the EDCBSCSS.

FIG. 4 shows a top and side view of the EDCBSCSS and the way in which the front protective tube is connected.

FIG. 5 shows an isometric image of the EDCBSCSS connected to the tube holder and the way the front protective tube is uncoupled.

FIG. 6 shows an isometric image of the EDCBSCSS, with the cannula retracted, and the retracting system of the puncturing cannula is shown in detail.

FIG. 7 shows a lateral cut A-A in Which details of EDCBSCSS in a charged state are shown.

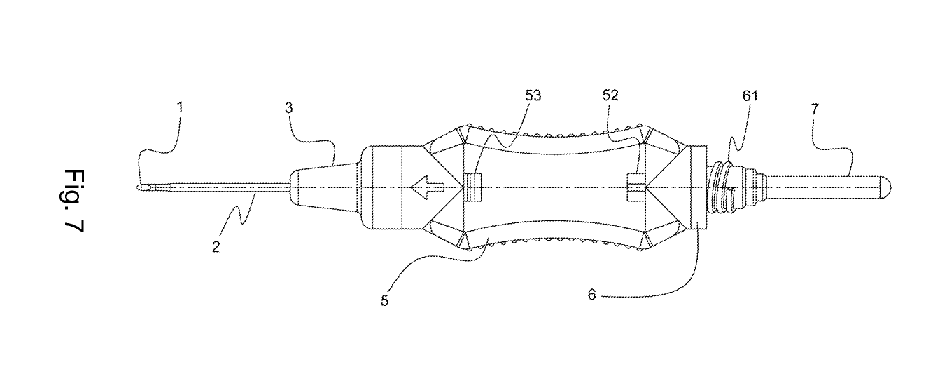

FIG. 8 shows a lateral cut A-A in which details of the EDCBSCSS in retraction and collecting of blood are shown.

FIG. 9 shows an isometric image of the space of the cap.

FIG. 10 shows an isometric image of the safety latch of the cannula holder.

FIG. 11 shows an isometric image of the set of safely latches of the cannula in their final configuration.

### DETAILED DESCRIPTION OF THE INVENTION

For the detailed description we will refer to the figures of the preferred version of the present invention.

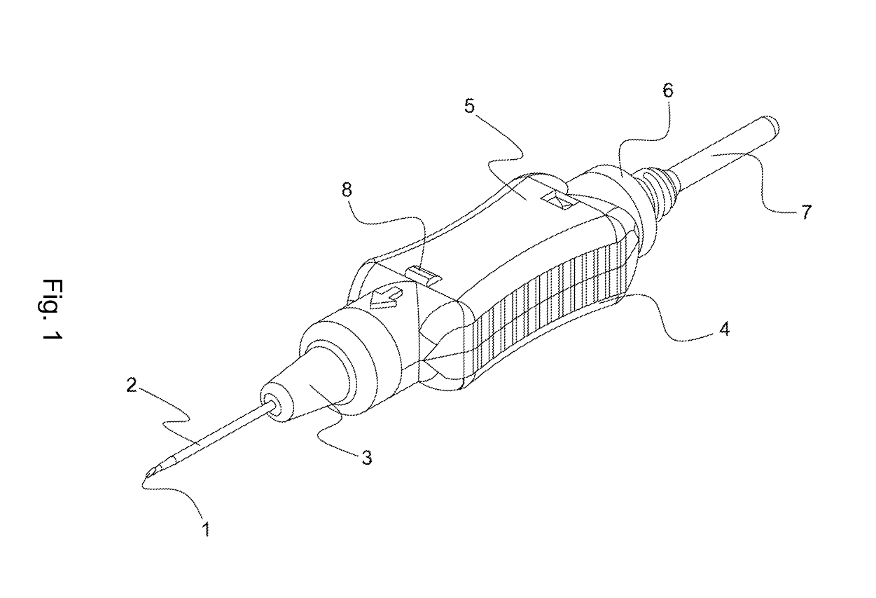

An isometric view of the Extraction device for collecting blood samples including a catheter and a safety system is shown in FIG. 1. The catheter (2) surrounds the puncturing cannula (1). The catheter (2) is connected to the catheter space (3) and this in turn is connected to the housing components (5) and (4). The puncturing cannula is connected to the safely latch (8) which is within the housings (5) and (4). At the opposite end of the catheter housing (3) the cap space (6) is connected. This is connected to the cap (7). A spring (9) can be found between the safely latch (8) and the puncturing cannula space (1) and this pushes the safety latch (8) once it is free, in order to retrieve the puncturing cannula (1) and this houses the extraction cannula (10) within the cap and through the cap space. The group of catheter space (3), cap space (6) and housing components (5) and (4) form a single unit when assembled.

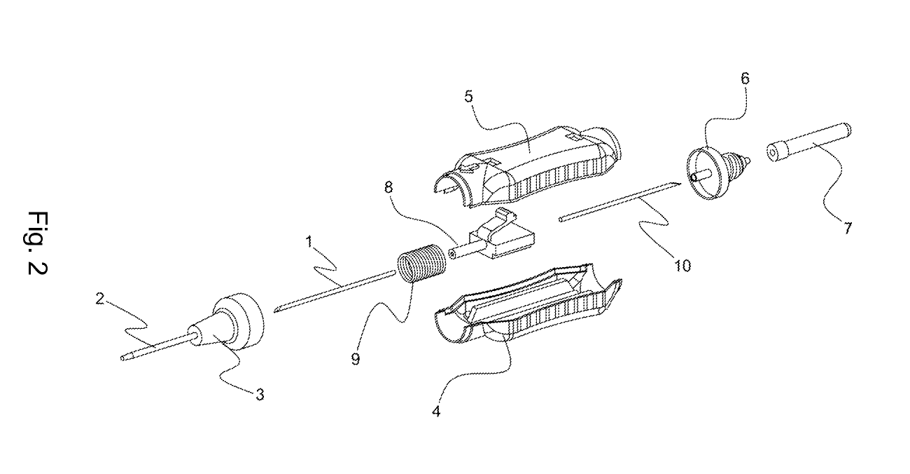

The EDCBSCSS consists of 10 elements as shown in the exploded view in FIG. 2. As may be seen in this FIG. 2, the parts which make up the EDCBSCSS are: (1) puncturing cannula; (2) catheter; (3) catheter space; (4) top housing; (5) bottom housing; (6) cap space; (7) cap; (8) cannula port safety latch; (9) spring and (10) collecting cannula.

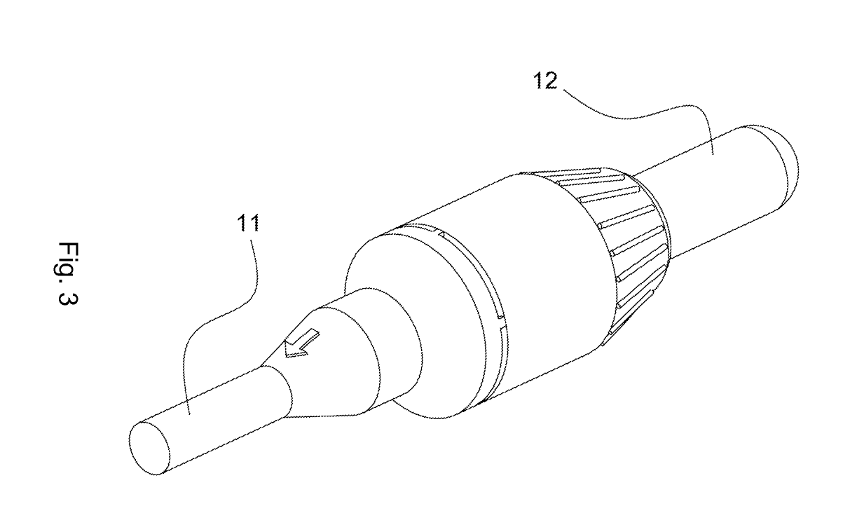

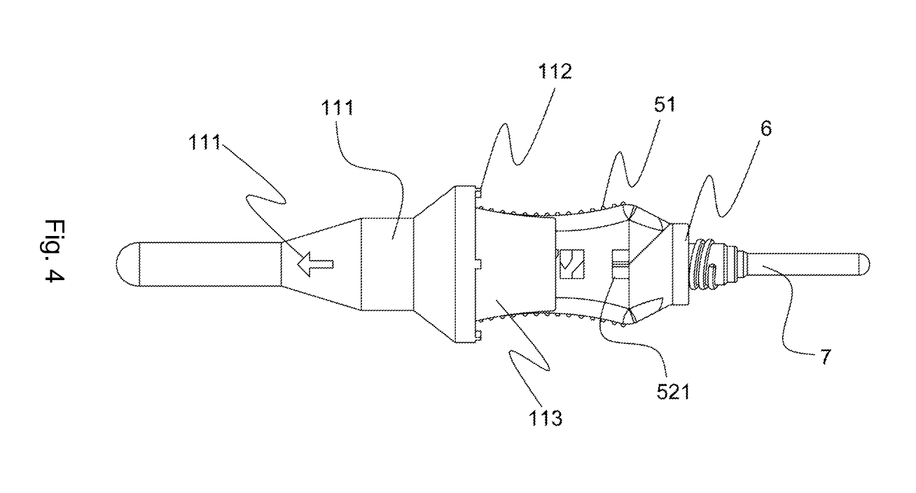

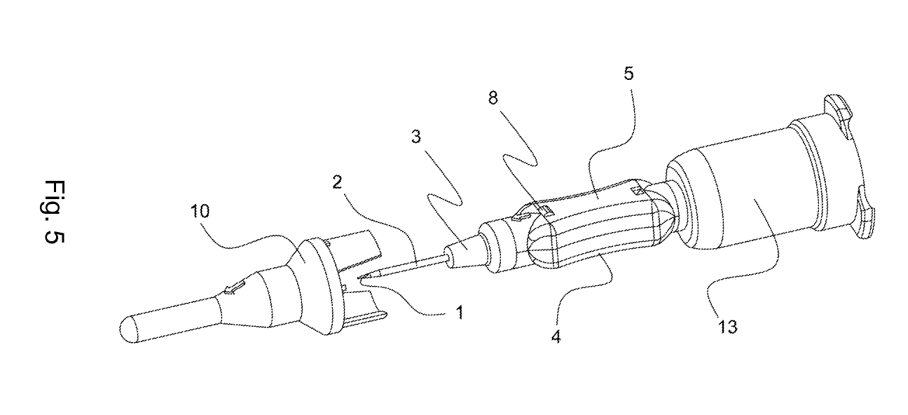

For transporting, the EDCBSCSS is housed in the protective tubes (11) and (12) shown in FIG. 3, whose function is to protect the EDCBSCSS during moving and prevent any contact at all with the needle before the EDCBSCSS is used. It is also equipped with the design characteristics necessary for sterilization. When the protective tube (12) is removed, part of the housings (4) and (5), the cap space (6) and the cap (7) are exposed, and only the front protector (11) remains in place, covering the catheter (2), puncturing cannula (1) and the catheter space (3), as show in FIG. 4. In this way the device is ready to be connected to the tube holder (13) and the EDCBSCSS is now ready for the puncturing and blood extraction procedure, as shown in FIG. 5. In FIG. 4 we can see details of the front protecting tube (11). These details ease the use of EDCBSCSS. The front protecting tube (11) present an indicator in the shape of an arrow (111) which lets the user know the direction and position of the tip of the puncturing cannula (1). Also, the front protecting tube has two wings (113) which connect to the rear protecting tube, leaving the handling curvatures (51) free, making it unnecessary to remove the front protecting tube until the procedure begins.

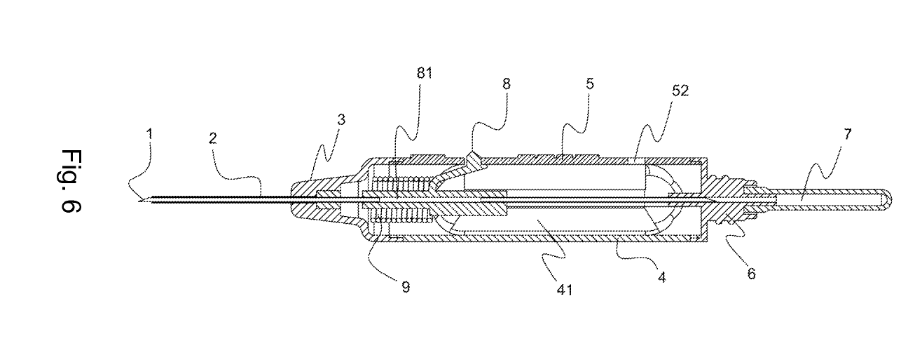

The detail of the EDCBSCSS in the charge state is shown in FIG. 6. The puncturing cannulas (1) and the collecting cannula (10) are linked to the safety latch (8) which is fixed inside the housings (4) and (5). This safety latch (8) is pushed by the spring (9) located between the safety latch (8) and the catheter space (3). In this charge state the puncturing cannula (1) is within the flexible catheter (2), while the collecting cannula is outside the rubber cap (7) but inside the pivot guide (63) of the cap space (6). In this position the EDCBSCSS is ready for the puncturing procedure and the placing of the catheter (2) in order to proceed with extracting the blood. Once the catheter (2) is in the vein, the retraction mechanism of the puncturing cannula (1) is activated and the collection cannula is ready for collecting the blood sample.

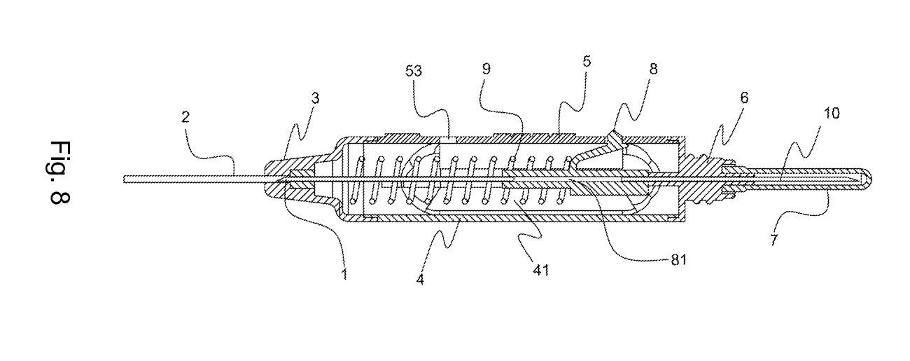

FIG. 8 shows the details of the EDCBSCSS in the retraction and blood collecting state. Once the puncturing has taken place and the catheter (2) and puncturing cannula (1) are in the patient's vein, the retraction mechanism of the EDCBSCSS may be activated. This is done by pushing the top housing (5) towards the head of the safety latch (82) which due to its geometric conditions will flex the neck of the safety latch (85) and disconnect the housing (5). At the same time it will be shifted due to the decompression of the spring (9). The safety latch (8) will shift within the housings (4) and (5) guided by the housing guides (41) until it connects by way of the safety latch head (82) with the rear anchoring slot (52), remaining fixed in this position. The puncturing cannula (1), upon being attached to the safety latch (8) will move together with it, so that only the catheter (2) remains inside the vein while the collecting cannula (7) joined to the safety latch (8) will slide inside the cap (7), habilitating the system for collecting blood. In the configuration the extraction of blood of the EDCBSCSS forms a flow channel made up of the catheter (2), puncturing cannula (1), safety latch flow channel (81) and extraction cannula (10). This channel presents the optimal geometric conditions for easy and efficient extraction of blood once the vacuum tube is connected to the extraction cannula (10).

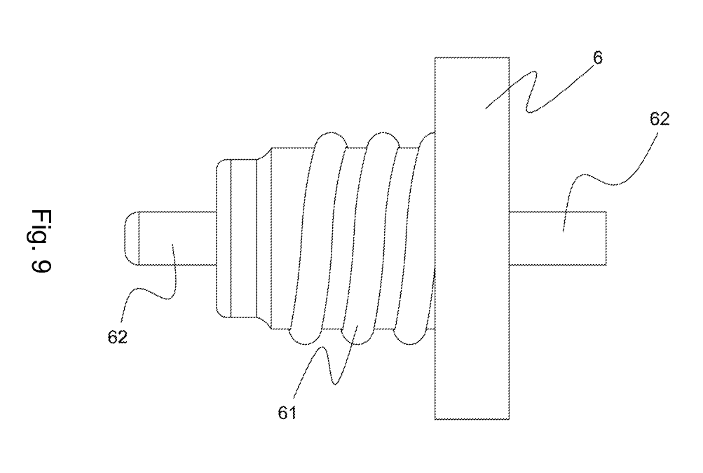

FIG. 9 shows an isometric image of the cap space (6), which connects to the components, cap (7), the collecting cannula (6) and the tube holder (13) which makes up the collection system of the flow channel of the EDCBSCSS. The cap space (6) has standard threads (61) which arc used to attach commercial tube holders for extracting blood samples, and also a hollow pivot on the cap (62) which is used to connect the cap (7), and on the opposite side there is a hollow pivot guide for the collecting cannula (63) in which the collecting cannula (7) is stored and guided when the system for retracting the puncturing cannula (1) is retracted. The cap space is joined to the housings (4) and (5) in such a way as to form a single body.

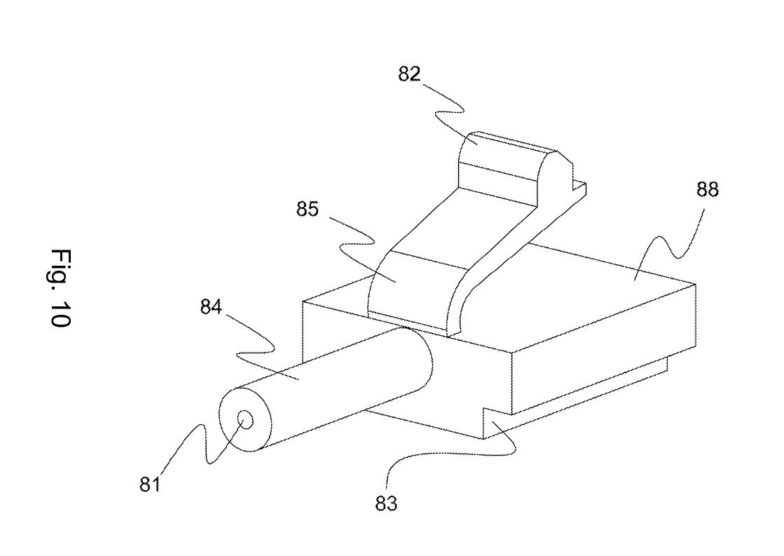

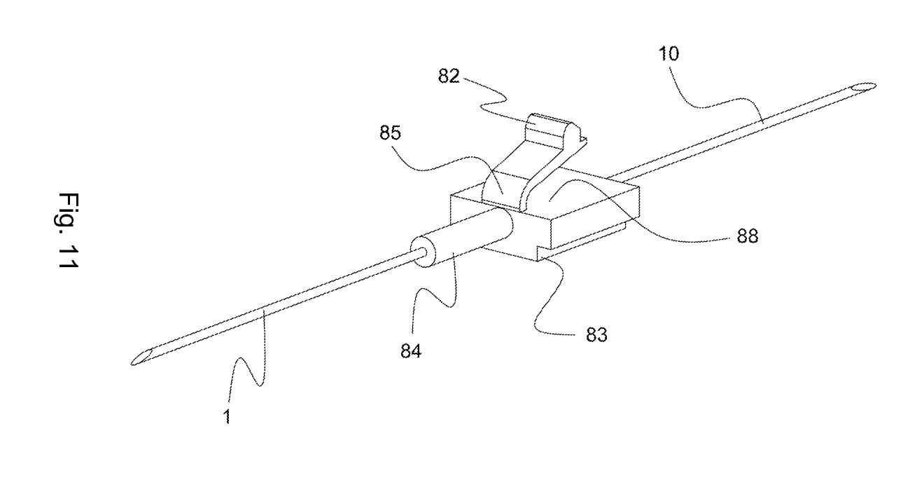

FIGS. 10 and 11 show details of the system for retracting the puncturing cannula of the flow channel of the EDCBSCSS. The safety latch (8) consists of a rectangular base (88) with two slide guides (83) in the lower part located on each side of the rectangular base (88). These guides along with the housing guides (41) control the direction of the displacement of the safety latch (8). On the top part of the rectangular base (88) a pliable neck (85) protrudes on whose tip is the head of the safety latch (82) whose function is to insert in the front anchoring slot (53) to conform the charge state of the EDCBSCSS. Once the reaction system is activated, the head of the safety latch (82) is inserted into the rear anchoring slit in order to activate the collection and safety system of the EDCBSCSS. On the front of the safety latch (8) a puncturing cannula pivot protrudes (84) and this supports and anchors the puncturing cannula (1) and acts as a guide for placing the spring (9). This pivot is hollow and constitutes a safety latch flow channel (81) which connects the puncturing cannula (1) to the collecting cannula (10) and together with the catheter (2) constitute the flow channel for extracting and collecting the blood sample towards the vacuum tube collector.

References

[1] Lavery I, Ingram P. Blood sampling: best practice. Nursing Standard, 2005, 19:55 65.

[2] Berkeris L et al. Trends in blood culture contamination. A College of American Pathologist Q-tracks study of 356 institutions. Archives of Pathology and Laboratory Medicine, 2005, 123:1222-1226.

[3] McCALL, R. E.; TANKERSLEY, C. M. Phlebotomy Essentials. Philadelphia, Lippincott William: Wilkins, 3rd ed., 2003.

The invention has been described sufficiently so that a person with knowledge in the field may reproduce and obtain the results, which we mention in the present invention. However, anyone skilled in the art of the present invention may be capable of making modifications not described in the present application. If for the application of these modifications in a determined structure or in the process of manufacturing, the matter claimed in the following claims is required, said structures are considered within the scope of the invention.

What we claim is:

1. An Extraction Device for Collecting Blood Samples, including a Catheter and a Safety System EDCBSCSS, characterized by consisting of a catheter (2) which is enclosed in a puncturing cannula (1). The catheter (2) surrounds the puncturing cannula (1). The catheter (2) is connected to the catheter space (3) and this in turn is connected to the housing components (5) and (4). The puncturing cannula is connected to the safely latch (8) which is within the housings (5) and (4). In the opposite end of the catheter housing (3) the cap space (6) is connected. This is connected to the cap (7). A spring (9) can be found between the safely latch (8) and the puncturing cannula space (1) and this pushes the safety latch (8) once it is free, in order to retrieve the puncturing cannula (1) and this houses the extraction cannula (10) within the cap and through the cap space.

2. The Extraction Device for Collecting Blood Samples, including a Catheter and a Safety System EDCBSCSS as claimed in the preceding claim, also characterized by the puncturing cannulas (1) and the collecting cannula (10) being linked to the safety latch (8) which is fixed inside the housings (4) and (5). This safety latch (8) is pushed by the spring (9) located between the safety latch (8) and the catheter space (3). In this charge state the puncturing cannula (1) is within the flexible catheter (2), while the collecting cannula is outside the rubber cap (7) but inside the pivot guide (63) of the cap space (6). In this position the EDCBSCSS is ready for the puncturing procedure and the placing of the catheter (2) in order to proceed with extracting blood. Once the catheter (2) is in the vein, the retraction mechanism of the puncturing cannula (1) is activated and the collection cannula is ready for collecting the blood sample.

3. The Extraction Device for collecting Blood Samples, including a Catheter and a Safety System EDCBSCSS, as described in claims 1 or 2 above, also characterized by consisting of a cap space (6) which is joined to the components, cap (7), the collecting cannula(6) and the tube holder (13) which form the collecting system of the EDCBSCSS.

4. The Extraction Device for Collecting Blood Samples, including a Catheter and a Safety System EDCBSCSS, as described in any of claims 1 through 3, also characterized by consisting of the top housing (5), the head of the safety latch (82), the neck of the safety latch (85) which when activated disconnect housing (5) and slide due to the decompression of the spring (9). The safety latch (8) will shift within the housings (4) and (5) guided by the housing guides (41) until it connects by way of the safety latch head (82) with the rear anchoring slot (52), remaining fixed in this position. The puncturing cannula (1), upon being attached to the safety latch (8) will move together with it, so that only the catheter (2) remains inside the vein while the collecting cannula (7) joined to the safety latch (8) will slide inside the cap (7) which constitute the system for blood collection.

5. The Extraction Device for Collecting Blood Samples, including a Catheter and a Safety System EDCBSCSS, as described in any of claims 1 through 4, also characterized by consisting of the safety latch (8) which consists of a rectangular base (88) with two slide guides (83) in the lower part located on each side of the rectangular base (88). These guides along with the housing guides (41) control the direction of the displacement of the safety latch (8). On the top part of the rectangular base (88) a pliable neck (85) protrudes on whose tip is the head of the safety latch (82) whose function is to insert in the front anchoring slot (53) to conform the charge state of the EDCBSCSS and also the head of the safety latch (82) is inserted into the rear anchoring slot in order to activate the collection and safety systems of the EDCBSCSS.

6. The Extraction Device for Collecting Blood Samples, including a Catheter and a Safety System EDCBSCSS, as described in any of claims 1 through 5, also characterized by the catheter space (3), the catheter housings (4) and (5), the cap space (6) and the cap (7) all joined together to form a single body.

7. The Extraction Device for Collecting Blood Samples, including a Catheter and a Safety System EDCBSCSS, as described in any of claims 1 through 6, also characterized by having a safety system in which housings (4) and (5) act simultaneously with the safety latch (8), the puncturing cannula (1) and the spring (9).
